(19) United States
(12) Patent Application Publication
(10) Pub. No.: US 2009/0031789 A1
(43) Pub. Date: Feb. 5, 2009

(54) LIQUID MEASURING DEVICE AND AUTOMATIC ANALYZER

(75) Inventor: Miyuki MURAKAMI, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS CORPORATION, Tokyo (JP)

(21) Appl. No.: 12/181,450

(22) Filed: Jul. 29, 2008

(30) Foreign Application Priority Data
Aug. 2, 2007 (JP) 2007-201879
Aug. 2, 2007 (JP) 2007-201880

(51) Int. Cl. G01N 29/00; G01N 33/48

(52) U.S. Cl. 73/61.49; 73/579; 356/39

(57) ABSTRACT

A liquid measuring device and an automatic analyzer which employs a resonance phenomenon of sound waves to detect an amount of a liquid reserved in a vessel with an opening are provided. The liquid measuring device includes a sound source that emits sound waves toward the opening, a control device that controls a frequency of the sound waves emitted by the sound source, a wave receiver that measures a resonance generated as the sound waves emitted by the sound source causes an air column in the vessel to resonate, a detector that detects a resonance frequency of the resonance measured by the wave receiver, and a liquid-amount determination unit that determines, based on the detected resonance frequency, an amount of the liquid reserved in the vessel.

### BACKGROUND OF THE INVENTION

1. Field of the Invention

The present invention relates to a liquid measuring device and an automatic analyzer.

2. Description of the Related Art

Conventionally, an automatic analyzer which analyzes a biological sample such as blood and bodily fluid uses a dispensing device to dispense a liquid sample such as a specimen and a reagent into a reaction vessel. In order to accurately dispense the liquid sample by a desired amount, the dispensing device obtains a volume of air inside a dispense nozzle when the dispensing device starts to dispense the liquid sample, and calculates, based on the obtained volume of air, an amount of a push-in operation of a plunger needed for dispensing the liquid sample by the desired amount (e.g., see Japanese Patent Application Laid-Open: No.2004-20320).

### SUMMARY OF THE INVENTION

A liquid measuring device of the present invention employs a resonance phenomenon of sound waves to detect an amount of a liquid reserved in a vessel with an opening, and includes a sound source that emits sound waves toward the opening, a control unit that controls a frequency of the sound waves emitted by the sound source, a measurement unit that measures a resonance of an air column inside the vessel caused by the sound waves emitted by the sound source, a detection unit that detects a frequency of the resonance measured by the measurement unit, and a liquid-amount determination unit that determines, based on the detected resonance frequency, an amount of the liquid reserved in the vessel.

Preferably, in the liquid measuring device of the present invention, in an aspect of the invention above, a sound-generation area of the sound source is larger than an area of the opening of the vessel.

Preferably, in the liquid measuring device of the present invention, in an aspect of the invention above, the control unit controls a driving signal of the sound source to modulate the frequency of the generated sound waves.

Preferably, the liquid measuring device of the present invention, in an aspect of the invention above, the control unit controls the driving signal of the sound source to generate sound waves whose frequencies differ from each other at the same time.

Further, an automatic analyzer of the present invention stirs a specimen and a reagent so that the specimen and the reagent are reacted, measures an optical property of a reaction liquid to analyze the reaction liquid, and detects, using the liquid measuring device, an amount of the specimen or the reagent dispensed into the vessel.

Preferably, the automatic analyzer of the present invention, in an aspect of the invention above, the liquid measuring device detects an amount of the dispensed specimen or the dispensed reagent after the specimen or the reagent are dispensed into the vessel.

Further, a liquid measuring device of the present invention employs a resonance phenomenon of sound waves to detect an amount of a liquid inside a vessel, and includes a measurement unit, which measures a resonance of an air column inside the vessel caused by sound waves emitted by a sound source, a reference-measurement unit, which measures a resonance of an air column inside a reference-vessel caused by the sound waves emitted by the sound source at the same time with the measurement unit, a detection unit, which detects a frequency of each resonance measured by the measurement unit and the reference measurement unit, a liquid-amount determination unit, which determines, based on each of the detected resonance frequencies, the amount of the liquid inside the vessel.

Preferably, the liquid measuring device of the present invention, in an aspect of the invention above, the liquid amount detection device includes a control unit, which controls a frequency of sound waves emitted by the sound source.

Further, an automatic analyzer of the present invention stirs a specimen and a reagent so that the specimen and the reagent are reacted, measures an optical property of a reaction liquid to analyze the reaction liquid, and detects, using the liquid measuring device according to claim 1, an amount of the specimen or the reagent dispensed into the vessel.

Preferably, the automatic analyzer of the present invention, in an aspect of the invention above, the liquid measuring device uses operation sound emitted by an operation unit of the automatic analyzer as sound waves emitted by the sound source.

Preferably, the automatic analyzer of the present invention, in an aspect of the invention above, the liquid measuring device detects an amount of the dispensed specimen or the dispensed reagent after the specimen or the reagent are dispensed into the vessel or the reference-vessel.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a schematic configuration diagram of a liquid measuring device and an automatic analyzer according to a first embodiment;

FIG. 2 is a schematic configuration diagram showing the liquid measuring device according to the first embodiment along with a reaction vessel; and

FIG. 3 is a diagram explaining a relation between a length of an air column of the reaction vessel and a wavelength of a resonance frequency;

FIG. 4 is a time-variation diagram of a frequency of sound waves that shows an example where a frequency of sound waves emitted toward the reaction vessel is changed;

FIG. 5 is a frequency characteristic diagram of an amplitude ratio of sound waves detected by a detector when sound waves whose frequency is changed from 10 to 100 kHz are emitted toward a reaction vessel into which 1 μL of a liquid is dispensed;

FIG. 6 is a frequency characteristic diagram of an amplitude ratio of sound waves detected by the detector when sound waves with changes in frequency within a range of 80 to 83 kHz are emitted toward each of reaction vessels into which a liquid is dispensed by a different amount;

FIG. 7 is a diagram explaining a detection timing at which a liquid amount is detected by the liquid measuring device;

FIG. 8 is a schematic configuration diagram showing a liquid measuring device according to a variation 1 along with a reaction vessel;

FIG. 9 is a schematic configuration diagram showing a liquid measuring device according to a variation 2 along with a reaction vessel;

FIG. 10 is a time-variation diagram of a frequency that shows another example where a frequency of sound waves emitted toward the reaction vessel is changed;

FIG. 11 is a variation diagram of a frequency that shows another example where a frequency of sound waves emitted toward the reaction vessel is changed;

FIG. 12 is a diagram showing, as an example, a resonance frequency of sound waves received by a wave receiver when the sound waves shown in FIG. 11 are emitted toward the reaction vessel;

FIG. 13 is a schematic configuration diagram showing a liquid measuring device according to a second embodiment;

FIG. 14 is a schematic configuration diagram showing a liquid measuring device and an automatic analyzer of the present invention;

FIG. 15 is a schematic configuration diagram showing the liquid measuring device of the present invention along with a reaction vessel;

FIG. 16 is a frequency characteristic diagram of an amplitude ratio of sound waves detected by the detector when sound waves with changes in frequency within a range of 80 to 83 kHz are emitted toward each of reaction vessels into which a liquid is dispensed by a different amount;

FIG. 17 is a schematic configuration diagram showing a variation 1 of the automatic analyzer of the present invention; and

FIG. 18 is a schematic configuration diagram showing a variation 2 of the automatic analyzer of the present invention.

### DETAILED DESCRIPTION OF THE PREFERRED EMBODIMENTS

### First Embodiment

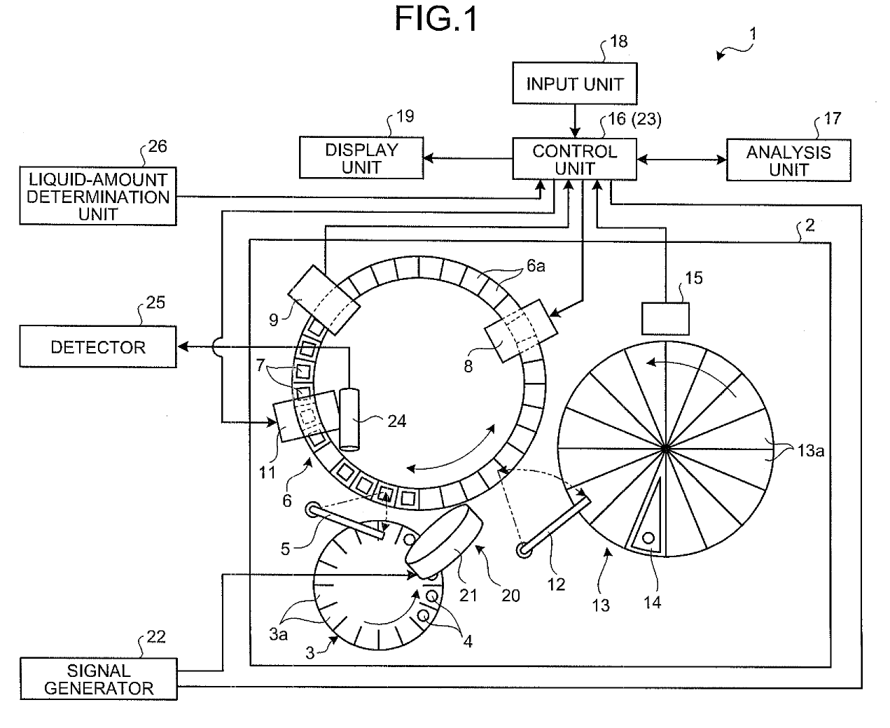

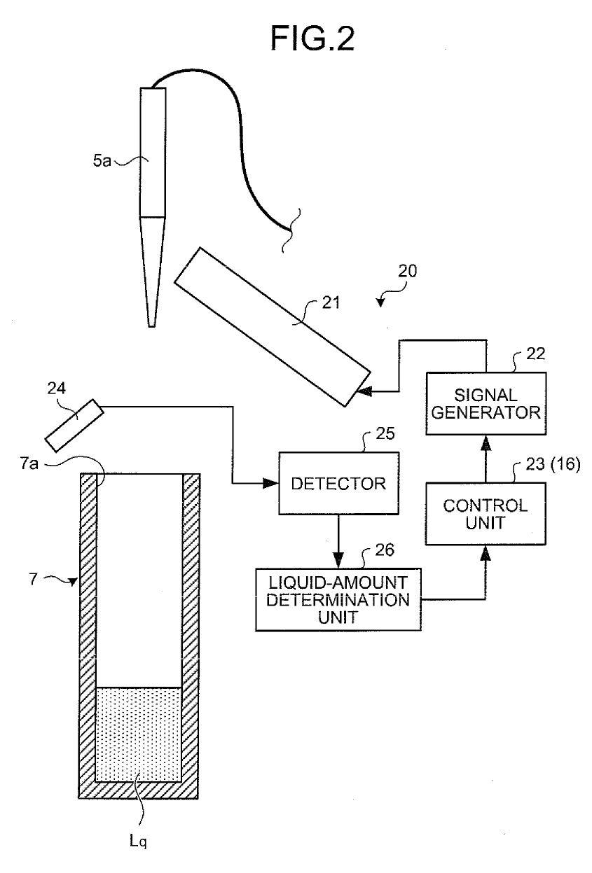

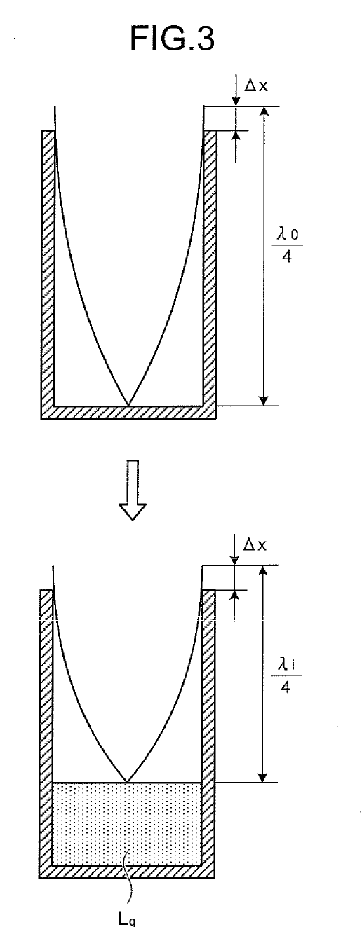

A first embodiment according to a liquid measuring device and an automatic analyzer of the present invention is described below in detail with reference to the drawings. FIG. 1 is a schematic configuration diagram of a liquid measuring device and an automatic analyzer according to a first embodiment. FIG. 2 is a schematic configuration diagram showing the liquid measuring device according to the first embodiment along with a reaction vessel. FIG. 3 is a diagram explaining a relation between a length of an air column of the reaction vessel and a wavelength of a resonance frequency.

In the automatic analyzer 1, as shown in FIG. 1, a specimen table 3, a specimen dispensing mechanism 5, a reaction wheel 6, a stirring device 8, a photometry device 9, a cleaning device 11, a reagent dispensing mechanism 12, and a reagent table 13 are arranged on a working table 2. A sound source 21 of a liquid measuring device 20 and a wave receiver 24 are arranged in the vicinity of the specimen dispensing mechanism 5.

The specimen table 3, as shown in FIG. 1, is rotated along a direction of the arrow by a driving unit, and on a periphery of the specimen table 3, plural storage rooms 3a are arranged at regular intervals in a circle. Each storage room 3a detachably stores therein a specimen vessel 4 containing a specimen.

The specimen dispensing mechanism 5 dispenses specimens into plural reaction vessels 7 stored in a reaction wheel 6. The specimen dispensing mechanism 5, as shown in FIG. 1, uses a dispense nozzle 5a (see FIG. 2) to sequentially suck the specimens out of plural specimen vessels 4 on the specimen table 3, and to discharge the sucked specimens into the reaction vessels 7, to thereby carry out a dispensing operation. In the specimen dispensing mechanism 5, a cleaning tank (not shown) which cleans an inside and an outside of the dispense nozzle 5a is arranged in a transfer pathway of the dispense nozzle 5a.

The reaction wheel 6 is rotated along the arrow shown in FIG. 1 by a driving unit different from the driving unit for the specimen table 3, and on a periphery of the reaction wheel 6, plural concave parts 6a are arranged at regular intervals in a circle.

In the reaction wheel 6, openings (not shown) through which measurement light transmits are formed at both sides of the radius of each concave part 6a. The reaction wheel is, for example, rotated in a counterclockwise direction by “(a circumference—a reaction vessel)/4” per one period, and is rotated in a clockwise direction by an amount of one concave part 6a per four periods. The stirring device 8, the photometry device 9 and the cleaning device 11 are arranged in the vicinity of the periphery of the reaction wheel 6.

The reaction vessel 7 is made from a transparent material which transmits more than or equal to 80% of analysis light emitted from a light source of the photometry device 9, for example, made of a synthetic resin such as glass including heat-resistant glass, cyclic olefin, and polystyrene. The reaction vessel having an opening 7a (see FIG. 2) at an upper end thereof is a tiny cuvette formed as a rectangular-cylindrical shape which can contain several μL to several hundreds μL. The reaction vessel 7 is arranged in the concave part 6a of the reaction wheel 6 with the facing sidewalls of the reaction vessel 7 being set along a radius direction.

Alternatively, the reaction vessel 7 can be formed as a cylinder shape to detect an amount of a reserved liquid as long as the reaction vessel 7 has an opening at one end.

The stirring device 8 is arranged on the reagent table 13 side in the vicinity of the periphery of the reaction wheel 6 The stirring device 8 stirs a liquid reserved in the reaction vessel 7 indirectly with the use of sound waves, or stirs the liquid directly with the use of a stir bar.

The photometry device 9 includes the light source, which emits analysis light for analyzing the liquid reserved in the reaction vessel 7, and a light receiver, which disperses and receives the analysis light transmitting through the liquid. In the photometry device 9, the light source and the light receiver are arranged at facing positions along a radius direction, sandwiching the concave part 6a of the reaction wheel 6.

The cleaning device 11 is arranged between the photometry device 9 in the neighborhood of the periphery of the reaction wheel 6 and the specimen dispensing mechanism 5. The cleaning device 11 includes a discharger which makes a liquid and a cleaning liquid discharge out of the reaction vessel 7, and a dispenser which dispenses the cleaning liquid. The cleaning device 11 makes the liquids on which the photometry has been performed, discharge out of the reaction vessel 7 after the photometry finishes, and then dispenses the cleaning liquid into the reaction vessel 7. The cleaning device 11 repeats the dispense operation and the discharge operation several times to clean an inside of the reaction vessel 7. The reaction vessel 7 cleaned as above is re-used for an analysis of a new specimen.

The reaction dispensing mechanism 12 dispenses reagents into plural reaction vessels 7 stored in the reaction wheel 6. As shown in FIG. 1, the reaction dispensing mechanism 12 dispenses the reagents in predetermined reagent vessels 14 on the reagent table 13 into the reaction vessel 7.

The reagent table 13 is rotated in the direction along the arrow shown in FIG. 1 by a driving unit different from the driving units for the specimen table 3 and the reaction wheel 6, and plural storage rooms 13a formed as a sector shape are arranged thereon in a circle. Each storage room 13a detachably stores therein the reagent vessels 14. The reagent vessels 14 are filled with the predetermined reagents meeting characteristics to be examined, and an information recording medium such as a bar-code label which indicates information about the contained reagent is attached to an outer surface of the reagent vessel 14.

On the periphery of the reagent table 13, as shown in FIG. 1, a reading device 15, which reads information recorded in the information recording medium attached to the reagent vessel 14, such as a type of the reagent, a lot, and an expiration date, and outputs the information to a control unit 16.

The control unit 16 is, for example, realized by a micro computer with a storage function which stores an analysis result and the like. The control unit 16 is connected to the specimen table 3, the specimen dispensing mechanism 5, the reaction wheel 6, the stirring device 8, the photometry device 9, the cleaning device 11, the reagent dispensing mechanism 12, the reagent table 13, the reading device 15, an analysis unit 17, an input unit 18, a display unit 19, the liquid measuring device 20, and the like. The control unit 16 controls an operation of each unit of the automatic analyzer 1. If the lot or the expiration date or the like of the reagent is not within a set range according to the information read from the recording of the information recording medium, the control unit 16 controls the automatic analyzer 1 to stop the analysis operation, or warns an operator.

The analysis unit 17 is connected to the photometry device 9 via the control unit 16. The analysis unit 17 analyzes a constituent concentration and the like of the specimen according to absorbance of the liquid in the reaction vessel 7 which is based on an amount of light received by the light receiver, and outputs the analysis result to the control unit 16. The input unit 18 performs an operation to input characteristics to be examined into the control unit 16, and is realized, for example, by a keyboard, a mouse, or the like. The display unit 19 displays a content of the analysis, a warning, and the like, and is realized by a display panel or the like.

The liquid measuring device 20 detects an amount of the liquid in the reaction vessel, employing a resonance phenomenon of sound waves. As shown in FIG. 2, the liquid measuring device 20 includes the sound source 21, a signal generator 22, a control unit 23, the wave receiver 24, a detector 25, and a liquid-amount determination unit 26.

The sound source 21 emits sound waves whose bundle has a cross-sectional area larger than the opening area of the reaction vessel 7 toward the opening 7a of the reaction vessel 7, and is realized, for example, by an airborne ultrasound device. As long as the sound source 21 can emit sound waves whose bundle has a cross-sectional area larger than the opening area of the reaction vessel toward the inside of the reaction vessel 7, the sound source 21 may be formed with a sound-generation area larger than the opening area of the reaction vessel 7, or the sound source 21 may be formed as an array of plural sound sources whose sound-generation area is smaller than the opening. Thus, the sound source 21 emits a bundle of sound waves whose cross-sectional area is larger than the opening toward the opening 7a of the reaction vessel 7. Therefore, the sound waves emitted by the sound source 21 are plane waves which cover over the opening 7a and transmit through the opening 7a into the reaction vessel 7.

Then, the resonance phenomenon of an air column that is closed at one end is caused in the reaction vessel 7 depending on the frequency of the emitted sound waves. A resonance frequency of sound waves which causes resonance of the air column is known to be inversely proportional to an amount of the retained liquid, that is, a length of the air column existing at upper portion of the liquid in the vessel. It is known that the length of the air column is one-fourth of a wavelength of the resonance frequency in a basic mode. For example, as shown in FIG. 3, when the reaction vessel 7 is empty, the length of the air column L0+Δx above the liquid is one-fourth of the wavelength λ0 of the resonance frequency f0 (f0=C/λ0, C: sound speed). When the length of the air column becomes Li+Δx as a liquid Lq is dispensed into the reaction vessel 7, the length of the air column Li is one-fourth of a wavelength λi (<λ0) of the resonance frequency in the basic mode (fi=C/λi, fi>f0). Δx is the length of the air column (open-end correction) which is above the opening of the tube.

Thus, the air-column length can be detected by detecting the frequency of resonance generated as the air column in the reaction vessel 7 resonates when the frequency of sound waves emitted toward the reaction vessel 7 is changed. Accordingly, with a previous measurement of the inner size of the reaction vessel 7, an amount of a dispensed liquid can be detected. That is the theoretical concept of the present invention adopts to detect the amount of the liquid dispensed into the vessel.

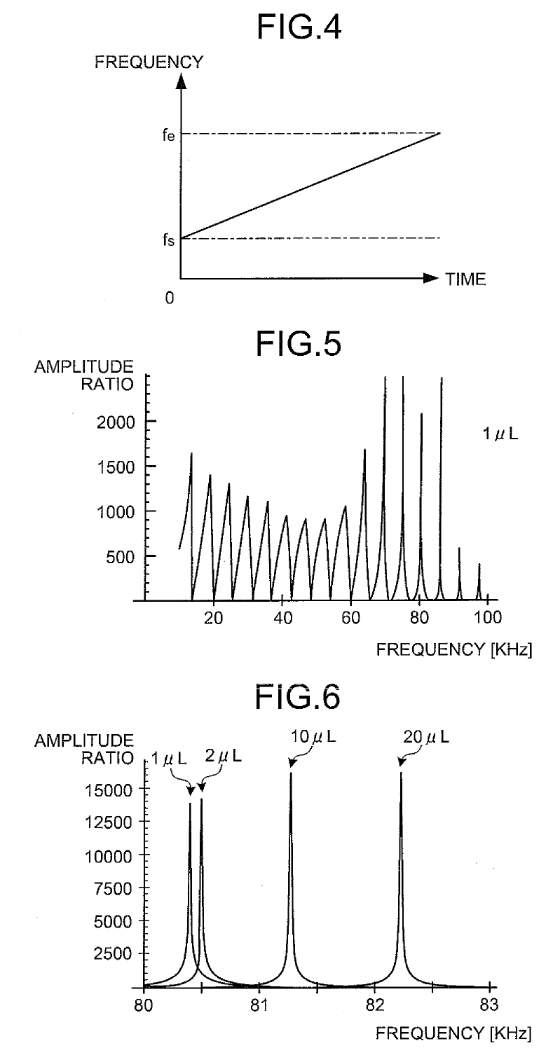

The control unit 16 of the automatic analyzer 1 works also as the control unit 23. The control unit 23 controls the signal generator 22 to control the frequency of sound waves emitted by the sound source 21 toward the reaction vessel 7. The control unit 23 controls the signal generator 22 to change the frequency of sound waves emitted by the sound source 21, and, for example, as shown in FIG. 4, the frequency is made to sweep from a frequency fs to a frequency fe in a certain period.

The wave receiver 24 receives waves of the resonance generated as sound waves emitted by the sound source 21 causes the air column in the reaction vessel 7 to resonate. The wave receiver 24 is realized, for example, by a wave receiver using an ultrasound device.

The detector 25 is a detection unit such as a signal analyzer, an FFT analyzer, and a multimeter, which detects sound waves emitted by the sound source 21. The detector 25 obtains an amplitude ratio P/Ar which is the ratio between an amplitude A of sound waves emitted by the sound source 21 toward the reaction vessel 7 and an amplitude P of resonance generated as the air column in the reaction vessel 7 resonates, to detect a resonance frequency, where the amplitude ratio P/A is the highest, that is, where the amplitude P of the resonance is largest.

The liquid-amount determination unit 26 determines the amount of the liquid reserved in the reaction vessel based on the resonance frequency detected by the detector 25, and is realized, for example, by a microcomputer, or the like. The liquid measuring device 20 measures in advance a relation between the resonance frequency and the amplitude ratio for each amount of the liquid dispensed into the reaction vessel 7 used in the automatic analyzer 1. Then, the liquid measuring device 20 stores the relations in the liquid-amount determination unit 26 as basic data.

The liquid-amount determination unit 26 compares the resonance frequency measured in a practical case for the liquid dispensed into the reaction vessel 7, to the basic data and determines the amount of the liquid. Thus, the liquid measuring device 20 can easily detect the liquid amount without considering the open-end correction. Alternatively, the liquid-amount determination unit 26 may determine the air-column length of the reaction vessel 7, which leads to the amount of the dispensed liquid, by calculation based on the resonance frequency measured in a practical case.

The automatic analyzer 1 of the present invention is configured as described above, and for example, the reagent dispensing mechanism 12 sequentially dispenses the reagents from the reagent vessels 14 into plural reaction vessels 7 transferred along a circular direction by the reaction wheel 6 rotating in the counterclockwise direction. The reaction vessels 7 into which the reagents have been dispensed are transferred along the circular direction by the reaction wheel 6. Then, the specimen dispensing mechanism 5 dispenses the specimens from the plural specimen vessels 4 stored in the specimen table 3 into the reaction vessels 7.

The reaction vessels 7 into which the specimens have been dispensed are transferred to the stirring mechanism 8 by the reaction wheel 6, the dispensed reagent and the dispensed specimen are sequentially stirred to be reacted. The reaction liquid where the specimen and the reagent have been reacted as above passes the photometry device 9 as the reaction wheel 6 rotates again, and there, the analysis light emitted by the light source transmits through the reaction liquid. Then, the photometry is performed on the reaction liquid of the reagent and the specimen in the reaction vessel 7 by the light receiver, so that the control unit 16 analyzes the constituent concentration and the like. Then, the reaction vessel 7 is cleaned by the cleaning device 11 after the analysis finishes, and is re-used for an analysis of a specimen.

The liquid measuring device 20 of the present invention can easily detect the amount of the liquid as below.

First, in the liquid measuring device 20, the control unit 23 controls the signal generator 22 to make the sound source 21 emit sound waves whose bundle has a cross-sectional area larger than the opening area of the reaction vessel 7 toward the opening 7a of the reaction vessel 7. The control unit 23 controls the signal generator 22 to change the frequency of sound waves emitted by the sound source 21 within a certain range.

Second, the wave receiver 24 measures the resonance generated as the emitted sound waves cause the air column in the reaction vessel 7 to resonate, and outputs a signal of the measured resonance to the detector 25. Third, based on the signal of the resonance input from the wave receiver 24, the detector 25 detects the frequency of the resonance. And, based on the resonance frequency detected by the detector 25, the liquid-amount determination unit 26 determines an amount of the liquid, and outputs the determined liquid amount to the control unit 23 as a liquid-amount signal.

For example, 1 μL of the liquid is dispensed into a cylinder-shaped reaction vessel having an opening with 6 mm diameter, and sound waves sweeping from 10 to 100 kHz are emitted toward the reaction vessel by the sound source 21, and the detector 25 detects the amplitude ratio P/A. As shown in FIG. 5, around 80 kHz, frequency characteristics showing a high resonance response to the frequencies are detected The peak of the amplitude ratio P/A indicates that the amplitude P of the resonance generated as the air column in the reaction vessel 7 resonates is the largest relative to the amplitude A of the emitted sound waves, and the frequency of each peak indicates the resonance frequency.

Based on the frequency characteristics, 1 μL, 2 μL, 10 μL, 20 μL of the liquid are respectively dispensed into the reaction vessels whose opening area is approximately same as that of the cylinder-shaped reaction vessel 7, and the amplitude ratio P/A is detected by the detector 25 with the frequency of the sound waves emitted by the sound source 21 sweeping from 80 to 83 kHz. As a result, as shown in FIG. 6, the reaction vessel 7 shows frequency characteristics where the resonance frequency, that is, the peak of the amplitude ratio P/A becomes increasingly high as the liquid amount increases. This result is consistent with the result of the air-column resonance in FIG. 3.

When the resonance frequency is detected by the liquid measuring device 20, frequency resolution and sensitivity to the liquid amount depends on the applied frequency range of sound waves. As shown in FIG. 5, however, the frequency range can be selected from plural frequency ranges. Therefore, the applied frequency range of sound waves are optimally selected corresponding to a specification such as an internal dimension of the reaction vessel 7, a dispensed amount, and a price of liquid measuring device 20. For example, when audible low-frequency range is selected and applied as the frequency range of sound waves, the liquid measuring device 20 can use a speaker as the sound source 21, and a microphone or the like as the wave receiver 24, whereas when high-frequency range is selected, the frequency resolution and accuracy of the liquid-amount detection can be enhanced.

As described above, the liquid measuring device 20 can easily detect the amount of the liquid dispensed into the reaction vessel 7 When the liquid amount detected by the liquid measuring device 20 based on the resonance frequency is not within the predetermined range, the automatic analyzer 1 may display on the display unit 19 a warning indicating that there is a problem in the liquid amount under a control by the control unit 23.

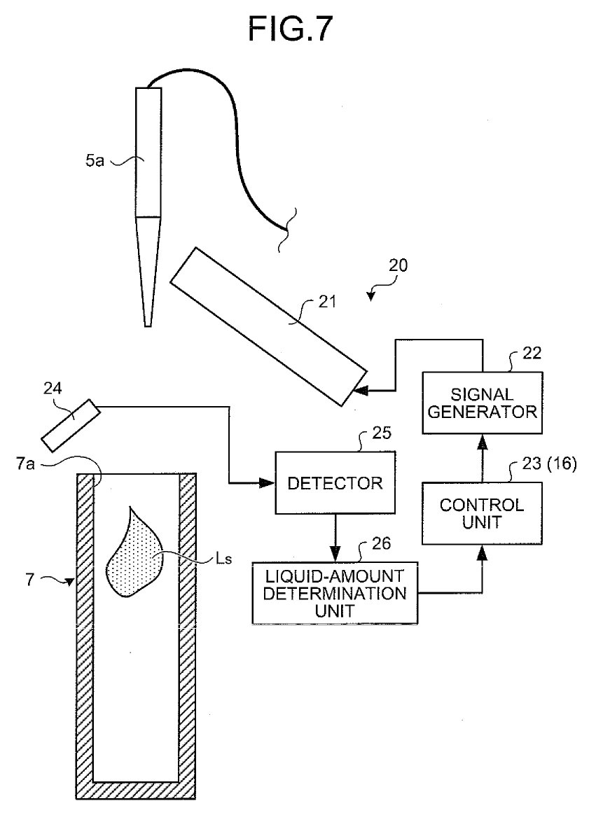

Further, the liquid measuring device 20 usually detects the liquid amount of the liquid which have been dispensed into the reaction vessel 7. However, as long as the detected liquid is below the opening of the reaction vessel 7, the detection device 20 can detect the amount of the partially-dispensed liquid, for example, a partially-dispensed specimen Ls, as shown in FIG. 7.

### Variation 1

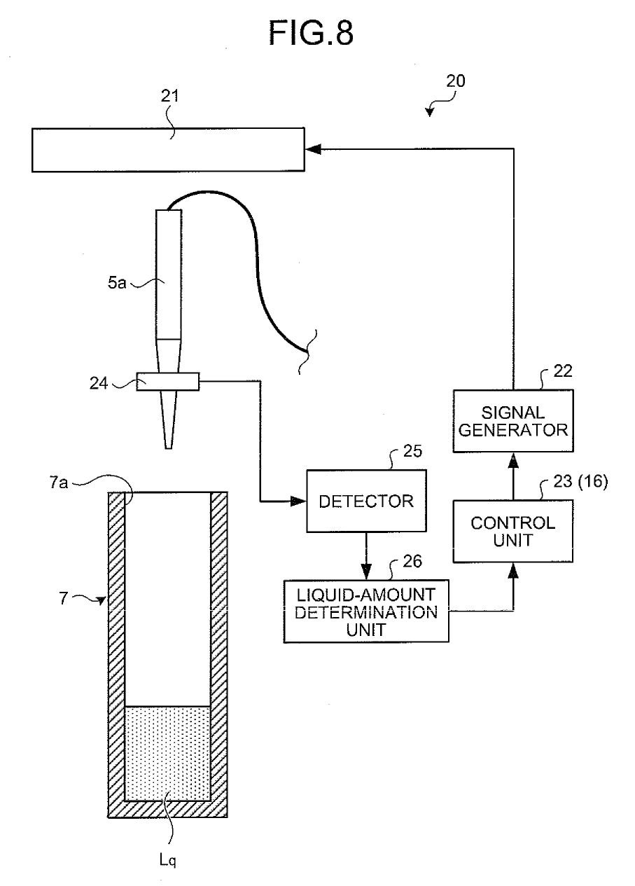

As shown in FIG. 8, in the liquid measuring device 20, the sound source 21 may be made larger than the same in the first embodiment, and the sound source 21 may be arranged above the dispense nozzle 5a to emit sound waves vertically from up above, and the wave receiver 24 may be attached to and integrated into the dispense nozzle 5a. With the arrangement above, since the sound source 21 is larger enough than the dispense nozzle 5a and the wave receiver 24, thus, the effect of sound wave diffraction is limited, even though the dispense nozzle 5a and the wave receiver 24 is in the sound path.

### Variation 2

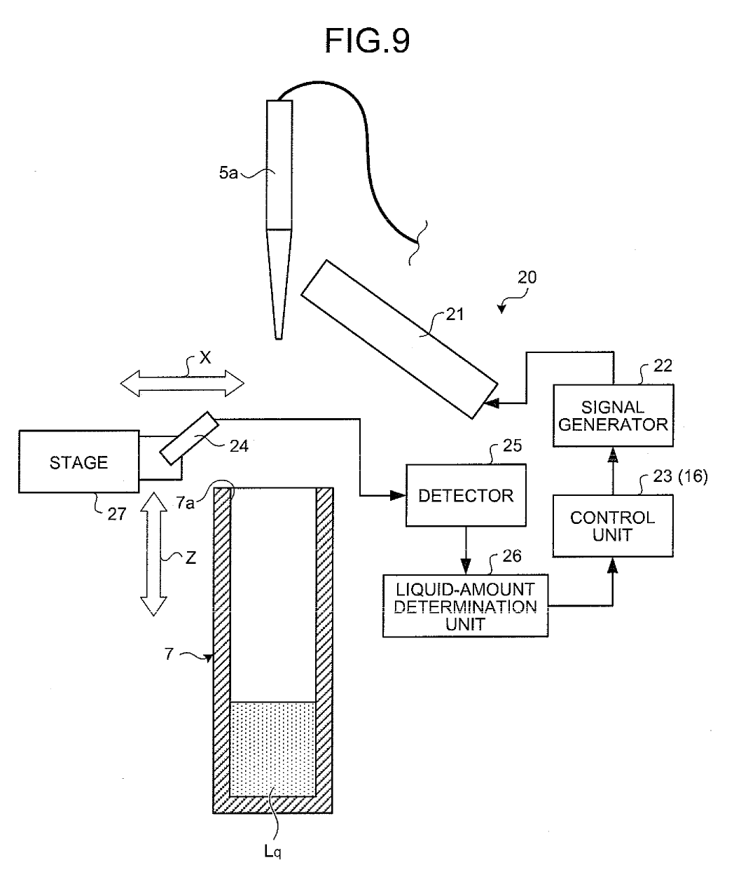

Further, as shown in FIG. 9, in the liquid measuring device 20, the wave receiver 24 may be arranged on a stage 27 in such a manner that the stage 27 allows the wave receiver 24 to move in a horizontal direction along the arrow X and in a vertical direction along the arrow Z. With the configuration above, the liquid measuring device 20 can move the wave receiver 24 to the most desirable position to receive the resonance generated by the resonating air column in the reaction vessel 7 depending on the dispensed amount of the liquid Lq dispensed into the reaction vessel 7.

The liquid measuring device 20 is arranged in the neighborhood of the specimen dispensing mechanism 5 to detect the amount of the liquid dispensed into the reaction vessel 7. However, the liquid measuring device 20 may be arranged in the neighborhood of the reagent dispensing mechanism 12 to detect the liquid amount of the reagent dispensed into the reaction vessel 7, or to detect the liquid amount of the reagent in the reagent vessel 14,

Further, when the sound source 21 changed the frequency of sound waves emitted toward the reaction vessel 7, the control unit 23 changed the frequency of sound waves by sweeping the predetermined range of the frequency. Alternatively, as shown in FIG. 10, the control unit 23 may change the frequency of sound waves emitted toward the reaction vessel 7 in such a manner that the frequency of sound waves is modulated from the frequency fs to the frequency fe in a certain cycle.

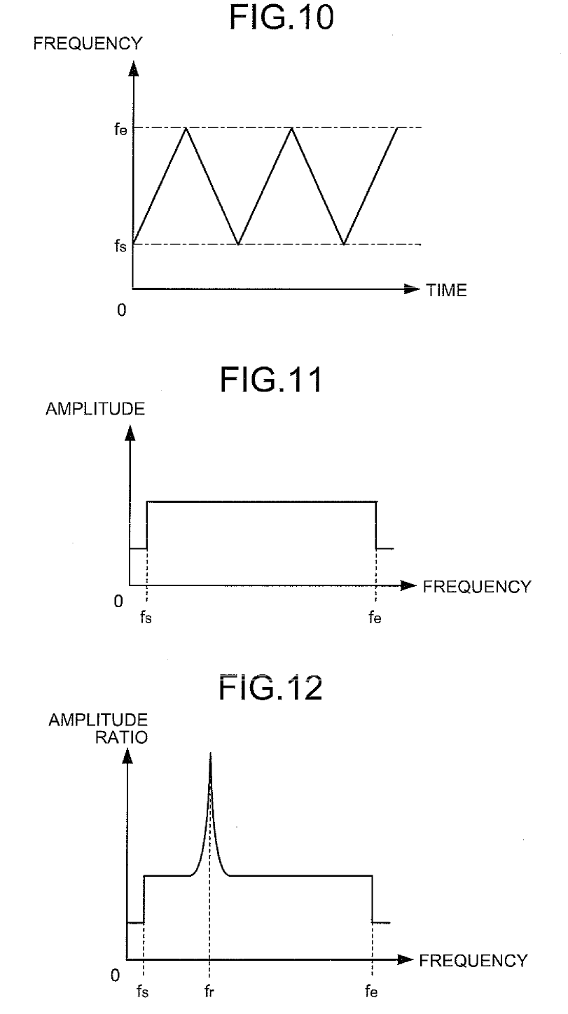

Further, as shown in FIG. 11, the control unit 23 may emit sound waves whose amplitudes are constant and whose frequencies differ from each other within a range between the frequency fs to the frequency fe at the same time, instead of the frequency modulation shown in FIG. 10. When the sound signal, which is outputted from the wave receiver 24 measuring the resonance generated by the resonating air column in the reaction vessel 7, is performed in fast Fourier transformation with a detector 25, the peak thereof is achieved at a position of the resonance frequency fr, as shown in FIG. 12.

In this case, the control unit 23 can shorten time needed for sweeping compared with the case where the frequency of sound waves sweeps within the predetermined frequency range.

The liquid-amount determination unit 26 obtains a vessel (air-column) length above the liquid in the reaction vessel 7, in other words, obtains a liquid-surface height of the liquid, based on the resonance frequency fr obtained by the fast Fourier transformation process. The liquid-amount determination unit 26 can easily obtain an amount of the dispensed liquid by multiplying the liquid-surface height with the inner size measured previously.

### Second Embodiment

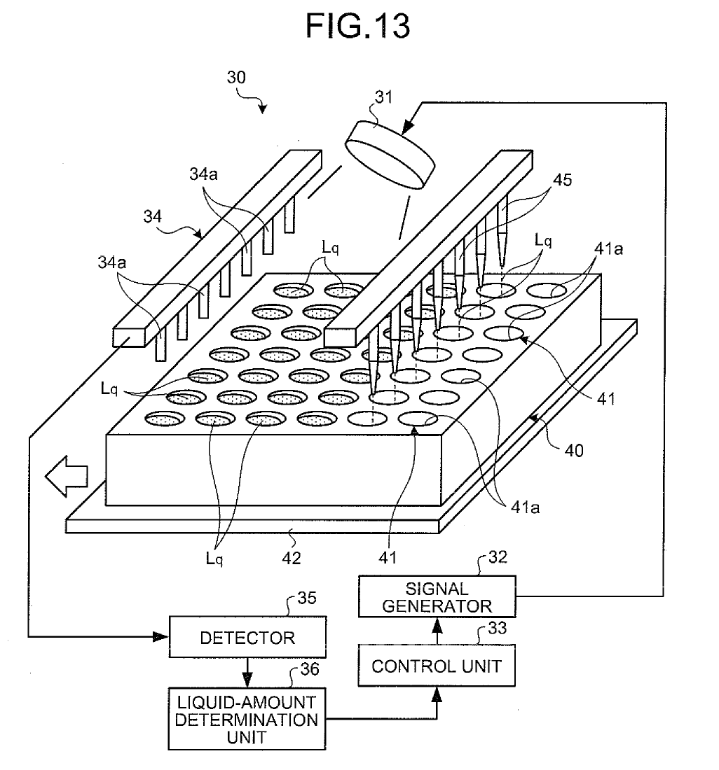

Next, a second embodiment according to the liquid measuring device of the present invention is described in detail with the drawings. The liquid measuring device according to the first embodiment detects the liquid amount of the liquid dispensed into the reaction vessel 7, whereas the liquid measuring device according to the second embodiment detects the liquid amount of the liquid dispensed into plural reaction vessels, each of which is called well, and formed on a microplate, and has an opening. FIG. 13 is a schematic configuration diagram of the liquid measuring device according to the second embodiment.

As shown in FIG. 13, the liquid measuring device 30 includes a sound source 31, a control unit 33, a wave-receiver unit 34, a detector 35, and a liquid-amount determination unit 36. A microplate 40 is a transparent plate made of a material such as acrylic resin. The microplate 40 has plural vessels 41 arranged in a matrix on a surface thereof, each of which is a concave unit having an opening 41a and is called a well. While the microplate 40 is transferred in a direction along the arrow by a transfer stage 42, a liquid Lq containing a reagent or a specimen is dispensed respectively into vessels 41 in each column from a corresponding dispense nozzle 45 of dispense nozzles 45.

The sound source 31 emits sound waves whose bundle has a cross-sectional area larger than an area of the opening 41a formed on the microplate 40 toward insides of the vessels 41 at the same time, and, for example, is realized by an airborne ultrasound device. As long as the sound source 31 can emit sound waves whose bundle has a cross-sectional area larger than the area of the opening 41atoward insides of the vessels 41 at the same time, a sound-generation area of the sound source 31 may be made larger than the area of the opening 41a. Thus, the sound waves emitted by the sound source 31 are plane waves which transmit into each vessel 41.

The control unit 33 controls a signal generator 32 to control a frequency of sound waves emitted by the sound source 31 toward the vessels 41. The control unit 33 controls the signal generator 32 to change the frequency of sound waves emitted by the sound source 31 toward the vessels 41, similarly to the control unit 23.

The wave-receiver unit 34 receives resonance, which is generated by air columns of the vessels 41 of each line with different frequencies emitted by the sound source 31, with each wave receiver 34a. Then, the wave receiver 34a outputs each of the waves individually to the detector 35 as a sound signal. The wave-receiver 34a is, for example, realized by a wave receiver using an airborne ultrasound device.

The detector 35 detects resonance frequency of sound waves which resonate in each vessel 4 based on the sound signals individually output by the wave receiver 34a. The detector 35 obtains an amplitude ratio P/A to detect a resonance frequency for each vessel 41. A size of the wave receiver and an interval of the openings 41a is configured to suppress an acoustic coupling (cross talk) among the adjacent vessels 41.

The liquid-amount determination unit 36 determines an amount of the liquid reserved in each vessel 41 based on the resonance frequency detected by the detector 35, and is realized, for example, by a microcomputer or the like. The liquid-amount determination unit 36 previously obtains a relation between the frequency and the amplitude ratio for each of amounts of the liquid dispensed into each vessel 41, and stores the relation therein as basic data. Then, the liquid-amount determination unit 36 compares the measured data of the frequency and the amplitude ratio, which are measured for a liquid sample dispensed into each vessel 41, with the stored basic data to determine a liquid amount of the liquid sample.

As described above, the liquid measuring device 30 can easily detect the amount of the liquid dispensed into the vessel based on the resonance frequency of sound in each vessel 41.

### Third Embodiment

A third embodiment according to the liquid measuring device and the automatic analyzer of the present invention is described below in detail with the drawings. The first and second embodiments uses a measurement unit to measure the resonance of the air column in the vessel whereas the third embodiment uses a measurement unit and a reference measurement unit to measure the resonance of the air column in the vessel.

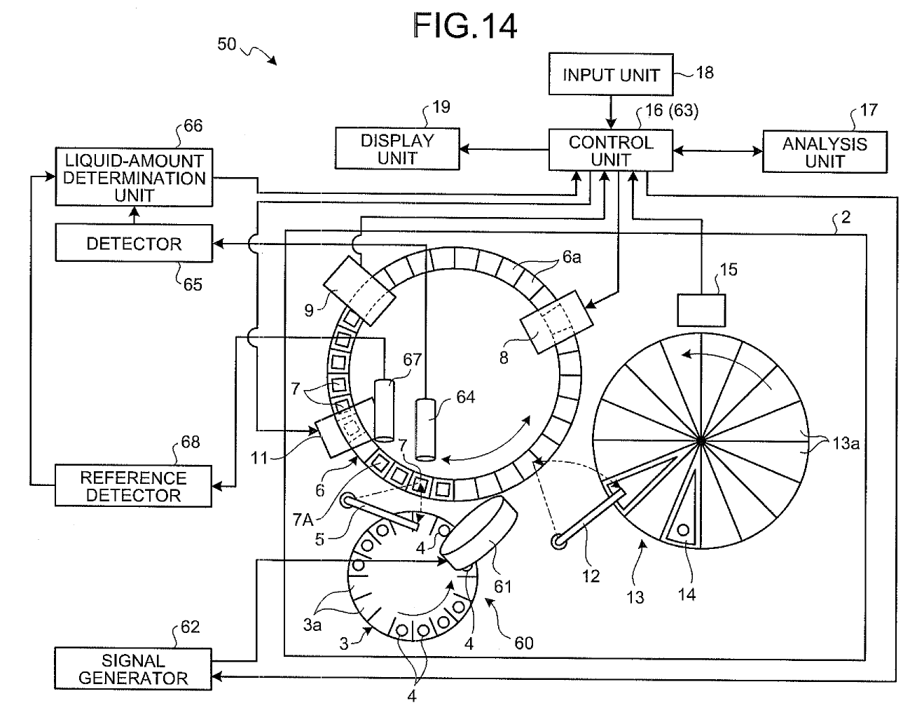

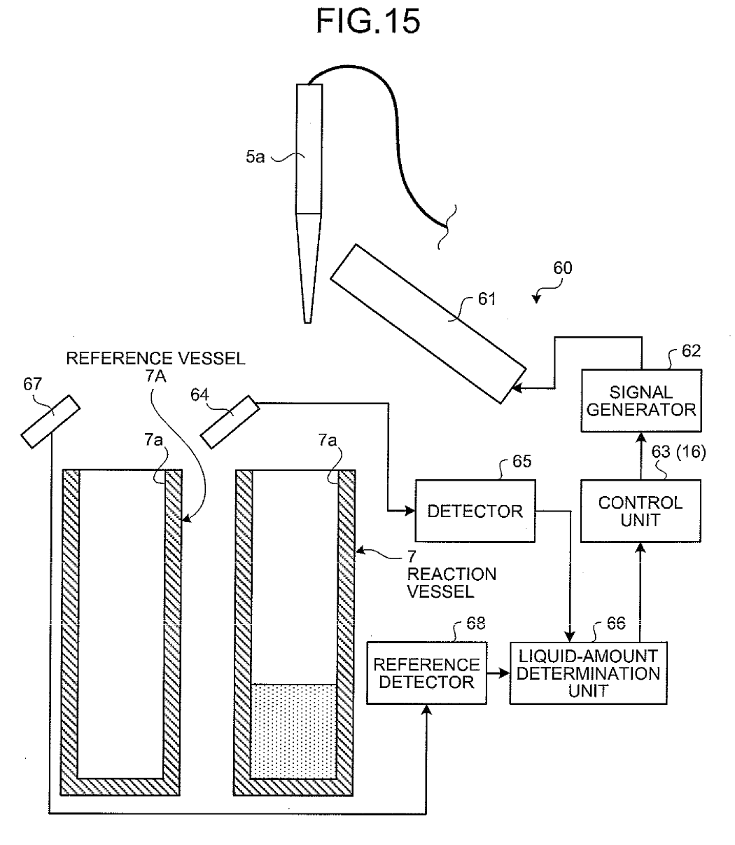

FIG. 14 is a schematic configuration diagram of a liquid measuring device and an automatic analyzer according to the third embodiment. FIG. 15 is a schematic configuration diagram showing the liquid measuring device according to the third embodiment along with a reaction vessel. In the automatic analyzer 50 according to the third embodiment, same numerals are attached to configurations which are identical with the automatic analyzer 1 except the automatic analyzer 50. As shown in FIG. 14, the automatic analyzer 50 includes an automatic analyzer 50 instead of a liquid measuring device 20.

The liquid measuring device 60 detects an amount of a liquid in the reaction vessel 7 employing a resonance phenomenon of sound waves. As shown in FIG. 15, the liquid measuring device 60 includes a sound source 61, a signal generator 62, a control unit 63, a wave receiver 64, a detector 65, a liquid-amount determination unit 66, a reference wave receiver 67, and a reference detector 68. The sound source 61, the signal generator 62, the control unit 63, the wave receiver 64, the detector 65, and the liquid-amount determination unit 66 are identical with the sound source 21, the signal generator 22, the control unit 23, the wave receiver 24, the detector 25, and the liquid-amount determination unit 26 of the automatic analyzer 1.

The liquid-amount determination unit 26 determines the amount of the liquid reserved in the reaction vessel 7 based on the resonance frequency detected by the detector 65 and the reference detector 68. Therefore, the liquid measuring device 60 measures in advance the resonance frequency of various liquid amounts dispensed into a reference vessel 7A and the reaction vessel 7, for the reaction vessel 7 used in the automatic analyzer 50, and stores a relation of the difference between the resonance frequency of the empty reference vessel 7A and the resonance frequency of the reaction vessel 7 with the liquid amounts, in the liquid-amount determination unit 66 as basic data.

The reference wave receiver 67 is the reference measurement unit which measures, in parallel with the wave receiver 64, the resonance generated as sound waves emitted by the sound source 61 causes the air column in the non-dispensed empty reaction vessel (referred to below as “reference vessel”) to resonate. The reference wave receiver 67 is realized by the same unit as the wave receiver 64.

The reference detector 68 is a reference detection unit to detect a frequency of the resonance measured by the reference wave receiver 67, and is realized by the same unit as the detector 65.

The liquid-amount determination unit 66 compares the difference between the resonance frequency detected by the detector 65 and the resonance frequency detected by the reference detector 68, to the basic data and determines the liquid amount of the liquid dispensed into the reaction vessel 7. The liquid measuring device 60 can easily detect the liquid amount without considering the open-end correction. Alternatively, the liquid-amount determination unit 66 may calculate the air-column length of the reaction vessel 7, that is, the amount of the dispensed liquid based on the resonance frequency of a practical measurement.

The automatic analyzer 50 is configured as above, and for example, the reagent dispensing mechanism 12 sequentially dispenses the reagents in the reagent vessels 14 into plural reaction vessels 7 transferred along a circular direction by the reaction wheel 6 rotating in the counterclockwise direction. The reaction vessels 7 into which the reagents have been dispensed are transferred along the circular direction by the reaction wheel 6. Then, the specimen dispensing mechanism 5 dispenses the specimens in the plural specimen vessels 4 stored in the specimen table 3 into the reaction vessels 7.

The reaction vessels 7 into which the specimens have been dispensed are transferred by the reaction wheel 6 to the stirring mechanism 8, where the dispensed reagent and the dispensed specimen are sequentially stirred to be reacted. The reaction liquid where the specimen and the reagent have been reacted as above passes the photometry device 9 as the reaction wheel 6 rotates again, and there, the analysis light emitted by the light source transmits through the reaction liquid. Then, the photometry is performed on the reaction liquid of the reagent and the specimen in the reaction vessel 7 by the light receiver, so that the control unit 16 analyzes the constituent concentration and the like. Then, the reaction vessel 7 is cleaned by the cleaning device 11 after the analysis finishes, and is re-used for an analysis of a specimen.

The liquid measuring device 60 can easily detect the amount of liquid in the reaction vessel 7 into which the specimen has been dispensed by using the resonance phenomenon of sound waves, as described below.

First, the liquid measuring device 60 controls the signal generator 62 by the control unit 63, and emits sound waves whose bundle has a cross-sectional area larger than a sum of opening areas of the reaction vessel 7 and the reference vessel 7A from the wave receiver 64 toward openings 7a of the reaction vessel 7 and the reference vessel 7A. At this time, the control unit 63 controls the signal generator 62 to change a frequency of sound waves emitted by the sound source 61 within a certain range.

Second, the wave receiver 64 and the reference wave receiver 67 measures resonances generated as the emitted sound waves causes the air column in the reaction vessel 7 to resonate, and outputs signals of the measured resonances to the detector 65 and the reference detector 68, respectively. In the description above, the reference wave receiver 67 detects the resonance in parallel with the wave receiver 64. Then, the detector 65 and the reference detector 68 detect the frequency of the resonance based on the signals of resonance input from the wave receiver 64 and the reference wave receiver 67.

Third, the liquid-amount determination unit 66 determines the liquid amount of liquid in the reaction vessel 7 based on a difference between the resonance frequency detected by the detector 65 and the resonance frequency detected by the reference detector 68, and outputs the determined liquid amount to the control device as a liquid-amount signal.

The specimen dispensing mechanism 5 dispenses 1 μL, 2 μL, 10 μL, and 20 μL of the liquid into the reaction vessels, respectively. The sound waves emitted by the sound source 61 sweeping from 80 to 83 kHz, and are coincidentally emitted toward the openings 7a of the reference vessel 7A (0 μL of liquid), into which the specimen is not dispensed, and the reaction vessel 7, into which the specimen has been dispensed, and then an amplitude ratio P/A is obtained by the detector 65 and the reference detector 68.

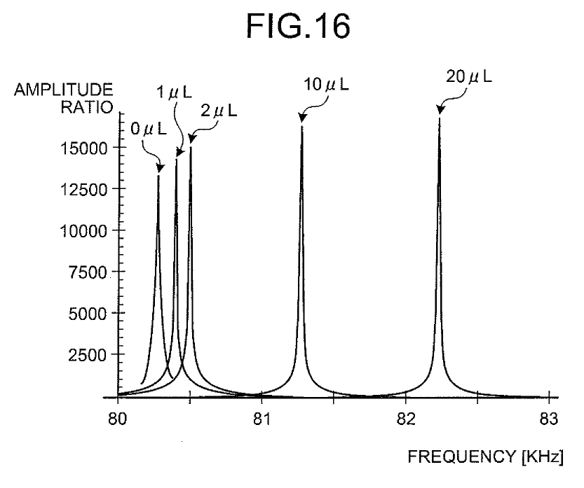

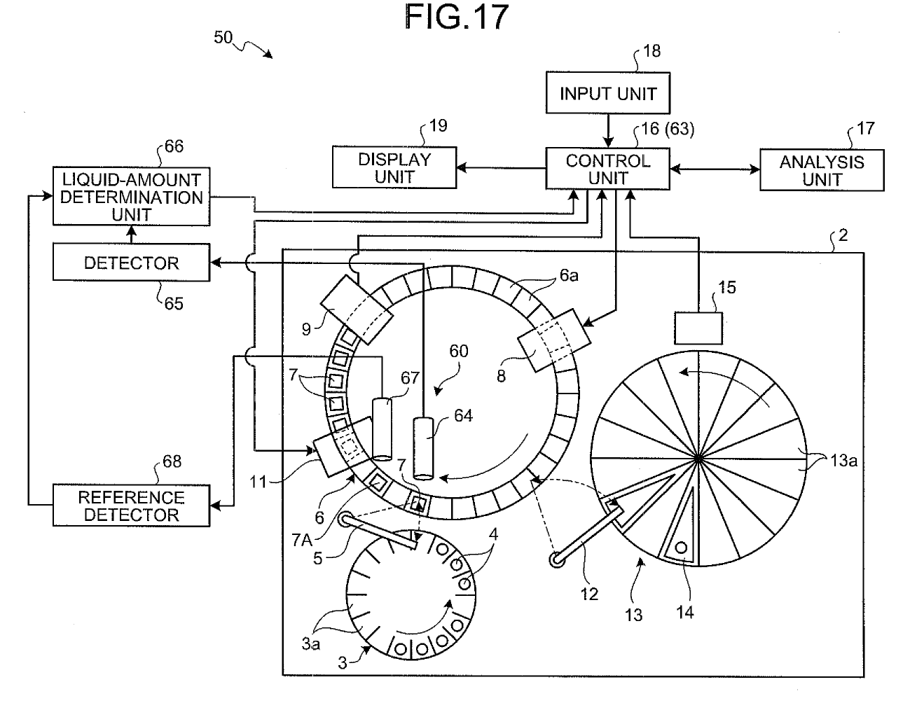

As a result, as shown in FIG. 16, the reaction vessel 7 shows frequency characteristics where the resonance frequency, that is, the peak of the amplitude ratio P/A becomes increasingly high as the liquid amount increases. This result is consistent with the result of the air-column resonance. The peak of the amplitude ratio P/A indicates that the amplitude P of the resonance generated by the resonating air columns in the reaction vessel 7 and the reference vessel 7A is the largest relative to the amplitude A of the emitted sound waves, and the frequency of each peak indicates the resonance frequency.

However, when the resonance frequency is detected by the liquid measuring device 60, frequency resolution and sensitivity to the liquid amount depends on the applied frequency range of sound waves, and the resonance of the air column occurs at intervals over plural frequency ranges. Thus, when the resonance frequency is detected by the liquid measuring device 60, the frequency range can be selected from plural frequency ranges. Accordingly, the frequency range is set at the range which is most suitable for the configuration such as inner sizes of the reaction vessel 7 and the reference vessel 7A, the dispense amount, and the price of the liquid measuring device 60. For example, when audible low-frequency range is selected and applied as the frequency range of sound waves, the liquid measuring device 60 can use a speaker as the sound source 61, and a microphone or the like as the wave receiver 64, whereas when high-frequency range is selected, the frequency resolution and accuracy of the liquid-amount detection can be enhanced.

As described, the automatic analyzer 50 including the liquid measuring device 60 can easily detect the amount of the liquid dispensed into the reaction vessel 7 with the use of the resonance phenomenon of sound waves. At this time, the wave receiver 64 and the reference wave receiver 67 measure in parallel the resonance generated by the sound waves emitted by the sound source 61. Accordingly, even when sound source 61 is irregular in time or in temperature such as the frequency of the emitted sound waves irregularly changes, or when environmental sound is not always constant, the wave receiver 64 and the reference wave receiver 67 measure resonance in parallel, that is, under a same measurement condition so that the difference between detection signals can be evaluated, whereby the liquid measuring device 60 can accurately detect the resonance frequency filtering out noise components.

When the liquid amount detected by the liquid measuring device 60 based on the resonance frequency is not within the predetermined range, the automatic analyzer 50 may display a warning indicating that there is a problem in the liquid amount on the display unit 19 under a control by the control unit 63.

### Variation 1

AS shown in FIG. 17, in the automatic analyzer 50, the liquid measuring device 60 may not include the sound source 61. In this case, the liquid measuring device 60 uses operation units, such as specimen table 3, the specimen dispensing mechanism 5, the reaction wheel 6, the stirring device 8, the cleaning device 11, the reagent dispensing mechanism 12, the reagent table 13 of the automatic analyzer 50, as a sound source, and then uses operational sound emitted by the operation units as sound waves emitted by the sound source. Thus, the liquid measuring device 60 does not require the signal generator 62 as well.

The automatic analyzer 50 emits operation sound within a wide range from audible sound to ultrasound. For example, the automatic analyzer for a biochemical analysis emits operational sound within a range of 50 Hz to 1 kHz. Therefore, with the use of the operational sound emitted by the operation units, the automatic analyzer 50 does not require the sound source 61 and the signal generator 62, whereby room-saving and cost reduction can be achieved.

In this case, the liquid measuring device 60 measures in parallel the resonance that is generated by the operation sound emitted by the operation units such as the reagent table 13 in the reaction vessel 7 and the reference vessel 7A with the wave receiver 64 and the reference wave receiver 67. Thus, even when the sound source 61 is irregular in time or in temperature, the liquid measuring device 60 can accurately detect the liquid amount in the reaction vessel 7 by detecting the resonance frequency. However, when the amplitude ratio is small depending the frequency of the operational sound emitted by the operation units as shown in FIG. 16, and the liquid measuring device 60 may be able to detect only whether there is any liquid left in the reaction vessel 7 or not.

### Variation 2

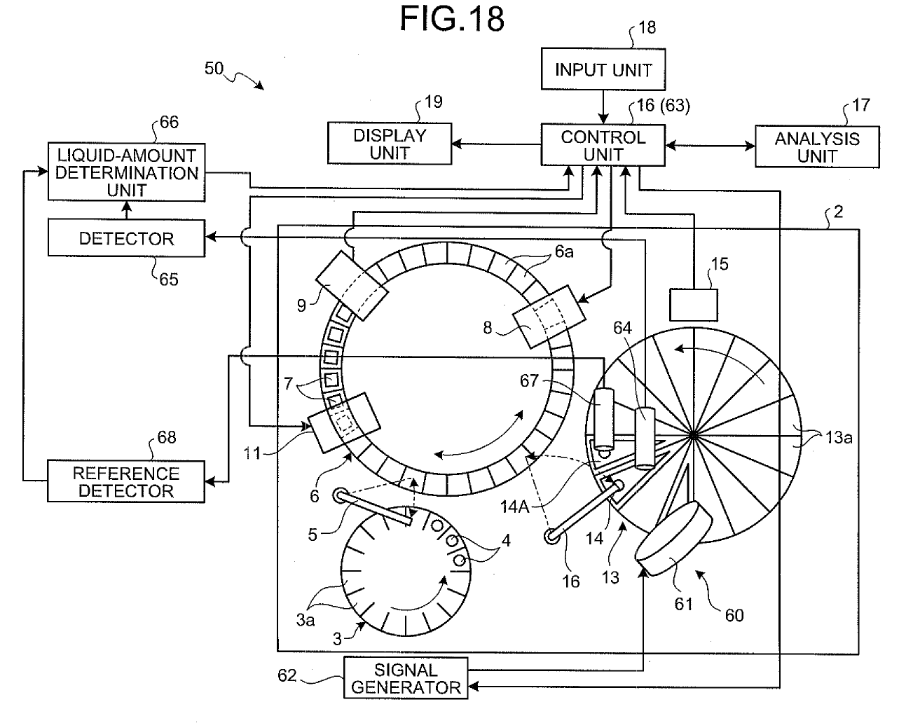

Further, as shown in FIG. 18, in the automatic analyzer 50, the sound source 61, the wave receiver 64, and the reference wave receiver 67 in the liquid-detection device 60 may be arranged in the vicinity of the reagent dispensing mechanism 12. And the remaining amount of the specimen may be detected thereby. In variation 2, a reference vessel 14A is arranged adjacent to the reagent vessel 14 with high consumption of reagent on the reagent table 13, and a remaining amount in the reagent vessel 14 is measured once in a predetermined number of rotations at a timing when the reference vessel 14A comes to a position of the reference wave receiver 67.

Further, the control unit 63 changes the frequency of sound waves emitted by the sound source 61 toward the reaction vessel 7 in such a manner that the frequency of sound waves sweep within a predetermined frequency range. Alternatively, the control unit 63 may change the frequency of sound waves emitted toward the reaction vessel 7 in such a manner that the frequency of sound waves is modulated within a range from a frequency fs to a frequency fe in a per cycle (see FIG. 10).

Further, the reference vessel 7A is described to be an empty vessel into which the liquid is not dispensed. Alternatively, the reference vessels 7A, 14A may be vessels reserving a predetermined amount of liquid.

Further, the liquid measuring device 60 includes two units, which are the detector 65 and the reference detector 68, as a detection unit for resonance. Alternatively, the detector may be one unit as long as the detector can distinguish the resonance measured by the wave receiver 64 and the resonance measured by the reference wave receiver 67.

Further, in the automatic analyzers 1 and 50 according to the first and the third embodiments, the reagent table 13 is described to be one unit, but may be two units instead. Further, in the automatic analyzers 1, 50, with a unit representing a device, plural units may be integrated into one unit.

1. A liquid measuring device which employs a resonance phenomenon of sound waves to detect an amount of a liquid reserved in a vessel with an opening, the liquid measuring device comprising:
a sound source that emits sound waves toward the opening;
a control unit that controls a frequency of the sound waves emitted by the sound source;
a measurement unit that measures a resonance of an air column inside the vessel caused by the sound waves emitted by the sound source;
a detection unit that detects a frequency of the resonance measured by the measurement unit; and
a liquid-amount determination unit that determines, based on the detected resonance frequency, an amount of the liquid reserved in the vessel.

2. The liquid measuring device according to claim 1, wherein a sound-generation area of the sound source is larger than an area of the opening of the vessel.

3. The liquid measuring device according to claim 1, wherein the control unit controls a driving signal of the sound source to modulate the frequency of the generated sound waves.

4. The liquid measuring device according to claim 2, wherein the control unit controls the driving signal of the sound source to modulate the frequency of the generated sound waves.

5. The liquid measuring device according to claim 1, wherein the control unit controls the driving signal of the sound to generate sound waves whose frequencies differ from each other at the same time.

6. The liquid measuring device according to claim 2, wherein the control unit controls the driving signal of the sound source to generate sound waves whose frequencies differ from each other at the same time.

7. An automatic analyzer which stirs a specimen and a reagent so that the specimen and the reagent are reacted, measures an optical property of a reaction liquid to analyze the reaction liquid, and detects, using the liquid measuring device according to claim 1, an amount of the specimen or the reagent dispensed into the vessel.

8. The automatic analyzer according to claim 7, wherein the liquid measuring device detects an amount of the dispensed specimen or the dispensed reagent after the specimen or the reagent are dispensed into the vessel.

9. A liquid measuring device which employs a resonance phenomenon of sound waves to detect an amount of a liquid inside a vessel, the liquid measuring device comprising:
a measurement unit that measures a resonance of an air column inside the vessel caused by sound waves emitted by a sound source;
a reference-measurement unit that measures a resonance of an air column inside a reference-vessel caused by the sound waves emitted by the sound source at the same time with the measurement unit;
a detection unit that detects a frequency of each resonance measured by the measurement unit and the reference measurement unit; and
a liquid-amount determination unit that determines, based on each of the detected resonance frequencies, the amount of the liquid inside the vessel.

10. The liquid measuring device according to claim 9, further comprising a control unit that controls a frequency of sound waves emitted by the sound source.

11. An automatic analyzer which stirs a specimen and a reagent so that the specimen and the reagent are reacted, measures an optical property of a reaction liquid to analyze the reaction liquid, and detects, using the liquid measuring device according to claim 9, an amount of the specimen or the reagent dispensed into the vessel.

12. An automatic analyzer which stirs a specimen and a reagent so that the specimen and the reagent are reacted, measures an optical property of a reaction liquid to analyze the reaction liquid, and detects, using the liquid measuring device according to claim 10, an amount of the specimen or the reagent dispensed into the vessel.

13. The automatic analyzer according to claim 11, wherein the liquid measuring device uses sound of an operation emitted by an operation unit of the automatic analyzer as sound waves emitted by the sound source.

14. The automatic analyzer according to claim 12, wherein the liquid measuring device uses sound of an operation emitted by an operation unit of the automatic analyzer as sound waves emitted by the sound source.

15. The automatic analyzer according to claim 11, wherein the liquid measuring device detects an amount of the dispensed specimen or the dispensed reagent after the specimen or the reagent are dispensed into the vessel or the reference-vessel.

16. The automatic analyzer according to claim 12, wherein the liquid measuring device detects the amount of the dispensed specimen or the dispensed reagent after the specimen or the reagent are dispensed into the vessel or the reference-vessel.

17. The automatic analyzer according to claim 13, wherein the liquid measuring device detects the amount of the dispensed specimen or the dispensed reagent after the specimen or the reagent are dispensed into the vessel or the reference-vessel.
